(19) United States
(12) Patent Application Publication
(10) Pub. No.: US 2012/0195911 A1
(43) Pub. Date: Aug. 2, 2012

(54) Method of treatment of cancer patients

(76) Inventors: Artur Martynov, Kharkov (UA); Boris S. Farber, Brooklyn, NY (US); Sonya Sophya Farber, New York, NY (US)

(21) Appl. No.: 12/931,465

(22) Filed: Feb. 1, 2011

(51) Int. Cl. A61K 39/42; A61P 35/00; A61P 31/12; A61K 31/522; A61K 38/21

(52) U.S. Cl. 424/159.1; 514/263.38; 424/85.7

(57) ABSTRACT

This invention may be used in human and veterinary medicine in combination with traditional methods of treatment of oncological illnesses for the purpose of increasing their effectiveness.

It is a method of treating patients with oncological diseases that is distinct in that an antiviral drug is used as an additional component of standard treatment before the beginning of and in parallel with standard treatment, in which Acyclovir, Valacyclovir, alpha interferon, a specific antiviral immunoglobulin or a combination of these substances are used as an antiviral drug, which may be used for inclusion in the standard surgical, radiological, or chemotherapeutic treatment plan for cancer patients.

The proposed method allows us to significantly improve the results of the treatment of cancer patients, decrease the number of relapses, improve patients' quality of life, and significantly extend their lives. Because the proposed drugs are already approved for use, there are no technical difficulties facing their inclusion in cancer patients' treatment plans.

### TECHNICAL FIELD

This invention is related to medicine—specifically, to oncology—and is intended to increase the effectiveness of the treatment of oncologic diseases in humans.

### PREVIOUS LEVEL OF TECHNOLOGY

Malignant neoplasms remain the scourge of economically developed countries, notwithstanding the successes in molecular biology and genetics of the past few decades. This is connected with a whole set of factors: the majority of carcinogens have a structure that is well-studied and functions that have been established, but the reason for the launch of carcinogenesis and the activation of the expression of carcinogens is not known (as a rule, several carcinogens are activated); the role of many carcinogenic viruses in the etiology of the cancerous process is known, but the reason for the cancerous transformation of cells under the influence of these viruses only in an insignificant part of cancer patients is not known; the mechanisms of the chemical and radiation carcinogenesis are known, but the reasons for that cancerous transformation in just an insignificant number of patients are unknown. There may be an endogenous cause facilitating the cancerous transformation. In our opinion, that factor is the combined persistence of several intracellular infective agents. The activation of several carcinogens in a cell at once is possible only when there is a whole other set of factors present that interferes with the cell's normal operations. These interfering factors are always focused on prolonging cell life, blocking apoptosis, and sending these infected cells out of control of the body's immune system. Which organisms benefit from infected cells going out of immune control? It is the intracellularly persistent agents that only transition into the active reproductive phase a few times a year and lie dormant inside the infective cells the rest of the time that are good for blocking immune response. The most widespread viruses from the herpesvirus, flu virus, papillomavirus, and adenovirus, persistent intracellular microorganisms like mycoplasms, chlamydia, legionelles, and great deals of other intracellular infective agents are types of persistent agents. The basic mechanism that prevents the liquidation of the infected cells by the immune system is the blocking of apoptosis through a variety of mechanisms.

In many cases, the viral persistence leads to the cancerous transformation of target cells. Like chemical carcinogenesis, this viral carcinogenesis is a multi-stage process. Cell transformation begins after infection of oncogenic viruses. In in vitro experiments, the next stage is characterized by the formation of a colony of cells with changed structure and multi-layered growth. These cellular properties are irreversible, and in many cases are not dependent on the presence of carcinogenic viruses inside them. As a rule, these cells may form in vivo tumors after subinoculation into syngen or athymus animals.

Carcinogenic viruses that are capable of playing the role of tumor-generating agents are represented as DNA and RNA viruses. For transformation of cells by DNA viruses an integration of the viral genome into the cell genome and the transfer of the changed genetic information to the cell's descendants must take place. Carcinogenic RNA viruses can also integrate their genomes into cell chromosomes, but initially, proviral DNA is synthesized with the participation of revertase in the infected cell in the RNA matrix. The synthesis of cell DNA is a required condition for the integration of viral DNA with cellular genes. The basic genes of carcinogenic viruses—viral carcinogens—take part in the initiation and support of the malignant transformation of cell targets. Upon stable transformation of cells, the carcinogen attaches itself to the genome and is in its activated state, while if there is an abortive transformation, the carcinogens function, but are not necessarily retained in the cell. The transformative effect of carcinogens is conditioned on the pleiotropic action of the proteins that are coded by these genes. This action may stimulate anomalously intensive cell division and (or) the blocking of cell apoptosis.

The discovery of endogenic viruses that are components of the genome of normal cells permitted a new interpretation of L. A. Zilber's viral genetic theory [¹]. However, questions on the meaning of the presence of endogenic viruses in the repressed (persistent) form and the possibility of their vertical transfer to descendants have not yet been answered. In connection with this, it is important to note the possibility of recombination between exo- and endogenic carcinogenic viruses, which allows defective sarcomatous viruses to complete their own reproductive cycle.

In the evolutionary process, two protective mechanisms formed in mammals that control the elimination from the organism of cells infected by viruses. The first of these is based on the immune reaction of the organism, which is directed against foreign viral proteins. This reaction manifests in the cytotoxic effect of special immune cells, which leads to the elimination of cells that have been transformed or infected by the virus. Cytotoxic T-lymphocytes fulfill their effector functions with the assistance of Fas ligand/Fas receptor systems and/or granzyme-perforin systems [²]. Another protective mechanism is connected with the activation of the cell cycle by the viral proteins in the infected cells [³]. Unlike that induced by cytokines, this activation ends in cell death through apoptosis [⁴].

The participation of the Epstein-Barr virus (EBV) in the development of infectious mononucleosis and its association with lymphoproliferative illnesses, nasopharyngeal and stomach cancer, and certain lymphomas, including Burkitt's lymphoma, has been established. In the latter case, the virus attacks circulatory B-lymphocytes exclusively [⁵] and the increased life spans of these cells is an important factor in viral persistence. The significance of the EBV in the formation of a malignant phenotype in lymphoma cells was demonstrated not long ago by J. Komano et al [⁶]. EBV—the positive cell line for Burkitt's lymphoma was selected by EBV—has negative clones that later underlay the infection by this virus. As it turns out, these clones (unlike the non-infectious EBV) are capable of growing in a semisolid agar medium and causing tumors in animals. The EBV-positive clones of the Burkitt's lymphoma cells demonstrated a significantly higher resistance to apoptosis than did the EBV-negative clones. Based on these data, the authors came to the conclusion that in order for the development of a malignant phenotype and resistance to apoptosis, the constant presence of EBV is required in Burkitt's lymphoma cells.

Testing of the action of the antiviral drug Cidofovir on the duplication of EBV-associated nasopharyngeal carcinomas in athymus mice established that this drug causes a quick induction of apoptosis in EBV-transformed epithelial cells [⁷]. The EBV's DNA shows up in the blood serum of patients with nasopharyngeal carcinoma [⁸], which also indicates its connection to the development of this disease.

The study of the molecular mechanisms of EBV-induced carcinogenesis presented the opportunity to discover a group of apoptosis-inhibiting proteins that take part in the formation and further development of the tumor process. The EBV's early antigen complex, BHRF1, stimulates B-lymphocytes infected with EBV to undergo the cellular cycle and survive [⁹] which can increase the infected cells' tendency to malignant transformation. Another protein, LMP1, which is coded by the EBV, demonstrates properties of a receptor that is capable of activating anti-apoptosis genes Bcl-2 and A20. It has been established that the anti-complementary oligonucleotides against the LMP1 gene slow proliferation, stimulate apoptosis, and increase the sensitivity of the B-lymphocytes immortalized by the EBV to the action of chemotherapy drugs [¹⁰].

Similarly to the Epstein-Barr virus, herpes simplex viruses types 1 and 2 (HSV-1 and -2), as well as herpesvirus saimiri, demonstrate properties of carcinogenic viruses. For example, it was discovered that transplantation of cells transformed by HSV-2 into athymus mice causes the formation of tumors in the animals [¹¹]. This virus demonstrates an affinity to cells in genital organs, while HSV-1 shows an affinity for mucus membranes of the lips and nasopharyngeal area, as well as to human skin integuments. It has been established that there is DNA fragmentation in cells infected by a mutated form of HSV-1 that lacks the α4 and Us3 genes (which code the main regulatory protein and the viral protein kinases respectively), while the “wild” (mutation-free) type of the virus does not have this effect [¹²]. Moreover, the “wild” type of the virus blocks human neuroblastoma cell apoptosis induced by the α-tumor necrosis factor using antibodies against Fas receptors, ceramides, or hyperthermia. A tissue specificity exists for the anti-apoptotic action of HSV-1 proteins. For example, human epidermal uterine carcinoma cells are resistant to this influence. The fact that the blockade of apoptosis of HSV-1-infected cells is not connected to its active reproduction is very important [¹³]. Unlike HSV-1, HSV-2 is capable of slowing the activity and level of Fas ligand expression in a cell membrane [¹⁴]. The infection of T-cells leads to the Fas ligand remaining hidden in the cell and not being expressed on cell plasmalemma. As a result, these cells lose that cytotoxic activity which is facilitated through Fas-dependent apoptosis.

The other carcinogenic virus, Herpes saimiri, codes protein ORF16, which is a functional analogue o the Bcl-2 protein [¹⁵]. As it turns out, a viral protein similar to Bcl-2 may create heterodimers with the pro-apoptotic Bak and Bax proteins, which results in the blocking of apoptosis induced by heterological viruses. For certain α-herpesviruses (and the verrucas planae virus), the production of special vFLIP anti-apoptotic proteins capable of cooperating with the FADD cell adaptor protein is inherent [¹⁶]. The survival of cells infected with these viruses facilitates the constant influence of interfering carcinogenic viruses that increase their transforming potential to a significant extent.

The tumor-forming activity of the human cytomegalovirus (HCMV) was recently demonstrated in vitro in experiments on primary cultures of the kidneys of embryonic rats [¹⁷]. The cancerous transformation of the cells evoked early HCMV genes IE1 and IE2, which, in combination with the gene from the E1A adenovirus, activated mutation in cell genes. It has been proven that the products of viral genes IE1 and IE2 can block apoptosis independent of one another [¹⁸]. IE proteins fulfill the function of transcription factors; the anti-apoptotic function of the IE2 protein is connected with the activation of the expression of cycline E (which is responsible for cells' transition to G1 in the S-phase of the cellular cycle) and the slowing of the post-transcription activity of the p53 protein [^{19,20}].

L. Burns et al [²¹] have established that the IE1 and IE2 viral proteins synergistically activate the expression of the ICAM-1 intercellular adhesion molecules in endothelial cells. The infection of neuroblastoma cells by the cytomegalovirus was accompanied by changes in their cytoskeletons and the level of expression of the integrated receptors, which increased the mobility of the cells and their dissemination [²²]. It is interesting that in long-term culturing of neuroblastoma cells infected by HCMV, they developed a resistance to the action of cysplatin and etoposide, although the viral DNA was not distinguished in them [19]. When the reproduction of the virus was blocked through treating the cells with Ganciclovir, their sensitivity to the action of antitumor drugs was fully restored. These data indicate that the infection of cells with cytomegalovirus before or in the process of tumor growth may increase their survival and the development of resistance to the action of anti-tumor drugs.

Thus the inclusion of antiviral drugs in the scheme for the treatment of malignant tumors and cardiovascular disease is justified.

A method for screening and treatment of oncological illnesses and diabetes is known [²³]. A sequence of special diagnostic procedures is applied if as a result of these procedures an oncological illness is diagnosed, treatment is conducted in the form of a combination of the Bacillus Calmette-Guerin (BCG) vaccination, oral administration of Valacyclovir twice a day and local application of Aldara cream.

The shortcoming of this method is its complex, expensive diagnostic procedure that does not correspond to standards, the impossibility of combining only Valacyclovir with standard schemes and methods of treatment or the necessity of the required combination of Valacyclovir with BCG vaccination and the application of cream based on imiquimod cream. The combination of Valacyclovir with standard methods of atherosclerosis treatment has not been provided for either.

### DISCLOSURE OF THE INVENTION

The invention's task was to increase the effectiveness of a method of treatment of oncological illnesses while taking into account the participation of the herpes virus in the etiology and pathogenesis of the abovementioned illnesses.

The task set is addressed through including Acyclovir drugs (injectible and tablet Acyclovir and Valacyclovir), other antiviral substances (specific antiviral immunoglobulins and alpha interferon) in the treatment plan for oncological illnesses: before and after surgical intervention, before and after radiation therapy, in combination with chemotherapy both individually and in standardized combination. Valacyclovir is taken in 1-2 g doses 3-4 times a day from 7-20 days in 3-7 courses. Acyclovir is administered intravenously in 0.5-1.0 g doses 3-4 times a day from 7-20 days in 3-7 courses as well.

This combination will lead to the rehabilitation of the immune system, expand the spectrum of antitumor activity of standard treatment methods, significantly increase the effectiveness of treatment of patients with adenocarcinomas with various localizations, and double to quintuple the remission period.

### BEST OPTION FOR INVENTION IMPLEMENTATION

### Example 1

### Preoperational Use in the Treatment of Mammary Gland Cancer

Patient L. 58 years old with a diagnosis of T2N1M0 mammary gland cancer, confirmed by a histological biopsy (a low-differentiated adenocarcinoma). Before treatment with Acyclovir, the antigen to cytomegalovirus (CMV) was found in 85% of the patient's lymphocytes through the fluorescing antibody method (FAM) and therapy was administered with the use of Valacyclovir. 1 g of medication was given 3 times a day for 7 days in a row in three courses in intervals of seven days each. Then the patient underwent a radical mastectomy. Postoperative chemotherapy was not used. No relapses were seen in the patient over a three-year period. The patient had a checkup every year and a study was done on the lymphocytes using the indirect immunofluorescence reaction (IIR) reaction for the presence of CMV. CMV antigens were not found in the lymphocytes.

### Example 2

### Preoperative Use of Valacyclovir in a Stomach Cancer Patient

Patient B. 62 years of age with a diagnosis of T3N1M0 stomach cancer confirmed histologically by biopsy (a low-differentiated adenocarcinoma), with metastasis in regional lymph nodes; CMV antigens were found using the FAM in 90% of the lymphocytes before treatment with Valacyclovir. The patient then underwent Valacyclovir therapy. 2 g of medication was administered 4 times a day for 7 days in a row in three courses in intervals of seven days each. Then a subtotal resection was performed on the patient's stomach. Postoperative chemotherapy was not used. No relapses were seen in the patient over a three-year period. Laboratory study of the patient's lymphocytes every year found CMV antigens in 10-15% of the cells. At the end of the first and second year, the patient received another course of Valacyclovir. Cancer did not reappear in the patient during the observation time.

### Example 3

### Use of Valacyclovir Before Radiation Therapy for Cervical Cancer

Patient S. 52 years of age with a diagnosis of T4N1M0 cervical cancer confirmed histologically through biopsy (a mid-differentiated adenocarcinoma) with massive invasion through all layers of surrounding tissue; CMV was found with the FEM in 80% of lymphocytes before treatment with Valacyclovir. The patient then underwent Valacyclovir therapy. 2 g of medication was administered 4 times a day for 7 days in a row in three courses in intervals of seven days each. Then the patient underwent a standard schedule of radiological treatment. Chemotherapy was not used. No relapses were seen in the patient over a three-year period. Laboratory study of the patient's lymphocytes every year found CMV antigens, but only in 20-30% of the cells. The patient underwent two more courses of treatment with Valacyclovir according to the schedule above once per year. Cancer did not reappear in the patient during the observation time, and there was no metastasis.

### Example 4

### Use of Valacyclovir Before Chemotherapy in Prostate Cancer Treatment

Patient D. 65 years of age with a diagnosis of T4N1M0 prostate cancer confirmed histologically through biopsy (a low-differentiated carcinoma); Valacyclovir was administered as treatment. CMV antigens were found in 95% of the patient's lymphocytes before treatment with Valacyclovir. 2 g of medication was administered orally 4 times a day for 7 days in a row in three courses in intervals of seven days each in parallel with CVPM chemotherapy. Other treatment methods were not used. No relapses were seen in the patient over a three-year period. Laboratory study of the patient's lymphocytes every year found CMV antigens in 10-15% of the cells. The patient underwent two more courses of treatment with Valacyclovir in combination with chemotherapy once per year. Cancer did not reappear in the patient during the observation time.

### Example 5

### Preoperative Use of Valacyclovir in a Stomach Cancer Patient

Patient S. 44 years of age with a diagnosis of T3N1M0 stomach cancer confirmed histologically by biopsy (a moderately differentiated adenocarcinoma), with metastasis in regional lymph nodes; EBV antigens were found 80% of the lymphocytes and CMV was found in 90% of the lymphocytes using the FAM in before treatment with Valacyclovir. The patient then underwent combination therapy with antiviral drugs: immunoglobulin to treat the EBV at a dosage of 13 ml of a 10% solution immediately (7 ml per injection) intravenously once a week three times; immunoglobulin to treat the CMV at a dosage of 13 ml of a 10% solution immediately (7 ml per injection) intramuscularly once a week three times; two 1 g tablets of Valacyclovir 3 times per day in 3 courses of 7 days each; Laferobion 3 million IU once per day daily for seven days in a row. Then a subtotal resection was performed on the patient's stomach. Postoperative chemotherapy was not used. No relapses were seen in the patient over a four-year period. Laboratory study of the patient's lymphocytes every year found CMV and EBV antigens in 10-15% of the cells. Cancer did not reappear in the patient during the observation time.

To prove the advantages of the method of treatment of oncological diseases with the use of Valacyclovir, 26 patients with cervical cancer, 18 patients with stomach cancer, and 12 patients with mammary gland cancer were studied who underwent Valacyclovir treatment before operations, radiation therapy, or chemotherapy. All the patients belonged to the second clinical group with stage 3 illness. In the capacity of a control group (Control), the following were used: 10 cervical cancer patients, 12 stomach cancer patients, and 10 mammary gland cancer patients from clinical group 2 with stage 3 illnesses. In the control group the patients were treated without the use of Valacyclovir and Acyclovir. Results of the study are presented in the table.

TABLE 1
The Comparative Characteristics of the
Results of Treatment of Cancer Patients

| | Method of Treatment of Cancer Patient | |
|---|---|---|
| | Experimental Group | Control |
| | Remission Period | Remission Period |
| Diagnosis | (weeks) | (weeks) |
| Cervical Cancer | 48* ± 5 | 16 ± 3 |
| Stomach Cancer | 42* ± 5 | 8 ± 2 |
| Mammary Gland Cancer | 45* ± 5 | 20 ± 7 |

*maximum observation period for experimental group: three years

As may be seen in the table, the remission periods due to the use of Valacyclovir (Acyclovir) before standard methods of treatment or in combination with chemotherapy doubled for mammary gland cancer and quintupled for stomach cancer. In the majority of the patients (more than 72%), metastasis was not seen for the entire period of observation (3 years). In the control group, only 12% of the patients had a remission time of over three years.

Thus the implementation of a new method of treatment of cancer patients with the use of Valacyclovir (Acyclovir) for pre-medication therapy or in combination with standard methods of treatment for adenocarcinoma permits:
facilitation of the rehabilitation of the immune system
expansion of the spectrum of antitumor activity for standard chemotherapy methods
an increase in treatment effectiveness for patients with stage 2 and 3 adenocarcinomas with various localizations
double to quintuple the remission period

Administering according to a plan of fewer than 3 courses of seven days each of less than 1 g 3 times a day for Valocyclovir perorally and Acyclovir at a dosage of less than 0.5 g 2 times a day in injected form does not provide long-term remission of the illness. The use of the antiviral drug is not useful for a longer period, as the remission period begins for the patient and the drug is ineffective.

### INDUSTRIAL APPLICABILITY

This invention is related to medicine—specifically to oncology—and may be used in cancer clinics for inclusion in the treatment complex for cancer patients with the goal of increasing the effectiveness of their treatment. All the proposed components are produced by the pharmaceutical industry and are accessible for use.

### REFERENCES

1 Zilber L A, Irlin I S, Kisilev F L. The Evolution of the Viral-Genetic Theory of Tumor Occurrence. Moscow: Science.-1975.-344 pp.
2 Podack E. R. Execution and suicide: cytotoxic lymphocytes enforce Draconian laws through separate molecular pathways.// Curr. Opin. Immunol.-1995.-Vol. 7.-P.11-16.
3 Prokhoryatova O V, Bila N V, Volkova V V, Martinov A V. A study of the interference of infectious laryngotracheitis viruses and Newcastle Disease in chicken embryos.// Veterinary Medicine.-2000.-Vol. 1, No. 78.- pp. 251-255.
4 Eick D., Hermeking H. Viruses as pacemakers in the evolution of defense mechanisms against cancer.//Trend. Genet.-1996.-Vol. 12.-P. 4-6.
5 Yao Q. Y., Ogan P., Rowe M., et al. Epstein-Barr virus-infected B cells persist in the circulation of acyclovir-treated virus carriers.// Int. J. Cancer.-1989.-Vol. 43.-P. 67-71.
6 Komano J., Sugiura M., Takada K. Epstein-Barr virus contributes to the malignant phenotype and to apoptosis resistance in Burkitt's lymphoma cell line Akata.// J. Virol.-1998.-Vol. 72.-P. 9150-9156.
7 Neyts J., Sadler R., De Clercq E, et al. The antiviral agent Cidofovir [(S)-1-(3-hydroxy-2-phpsphonyl-methoxypropyl) cytosine] has pronounced activity against nasopharyngeal carcinoma grown in nude mice.// Cancer res.-1998.-Vol. 58.-P. 384-388.
8 Matirangura A., Pornthanakasem W, Theamboonlers A, et. al. Epstein-Barr viral DNA in serum of patients with nasopharyngeal carcinoma.// Clin. Cancer Res.-1998.-Vol. 4.-P. 665-669.
9 Dawson C. W., Dawson J., Jones R., et al. Functional difference between BHRF1, the Epstein-Barr virus-encoded Bcl-2 homologue, and Bcl-2 in human epithelial cells.// J. Virol.-1998.-Vol. 72.-P 9016-9024.
10 Kenney J. L., Guinness M. E., Curiel T., Lacy J. Antisense to the Epstein-Barr virus (EBV)-encoded latent membrane protein 1 (LMP-1) suppresses LMP-1 and bcl-2 expression and promotes apoptosis in EBV-immortalized B cells.// Blood.-1998.-Vol. 92.-P.1721-1727.
11 Aurelian L. Herpes simplex virus type 2: unique biological properties include neoplastic potential mediated by the PK domain of the large subunit of ribonucleotide reductase.// Front Biosci.-1998.-Vol. 3.-P. 9237-9249.
12 Galvan V., Roizman B. Herpes simplex virus 1 induces and blocks apoptosis at multiple steps during infection and protects cells from exogenous inducers in a cell-type-dependent manner.// Proc. Natl. Acad. Sci. USA.-1998.-Vol. 95.-P. 3931-3936.
13 Koyama A. H., Miwa Y. Suppression of apoptotic DNA fragmentation in herpes simplex virus type 1-infected cells.//J. Virol.-1997.-Vol. 71.-P. 2567-2571.
14 Sieg S., Yildirim Z., Smith D, et. al. Herpes simplex virus type 2 inhibition of Fas ligand expression.// J. Virol.-1996.-Vol. 70.- P.8747-8751.
15 Nava V. E., Cheng E. H., Veliuona M, et. al. Herpesvirus saimiri encoded a functional homolog of the human bcl-2 oncogene.// J. Virol.-1997.-Vol. 71.- P.4118-4122.
16 Thome M., Schneider P., Hofmann K., et al. Viral FLICE-inhibitory proteins (FLIPs) prevent apoptosis induced by death receptors.// Nature.-1997.-Vol. 386.- P.517-521.
17 Shen Y., Zhu H., Shenk T. Human cytomegalovirus IE1 and 1E2 proteins are mutagenic and mediate “hit-and-run” oncogenic transformation in cooperation with the adenovirus E1 A proteins.//Proc. Natl. Acad. Sci. USA.-1997.-Vol. 94.-P. 3341-3345.
18 Zhu H, Shen Y, Shenk T. Human cytomegalovirus IE1 and 1E2 proteins block apoptosis.// J. Virol.-1995.-Vol. 69.- P. 7960-7970.
19 Bresnahan W A, Albrecht T, Thompson E A. The cyclin E promoter is activated by human cytomegalovirus 86-kDa immediate early protein.// J. Biol. Chem.-1998.-Vol. 273.-P. 22075-22082.
20 Vermeulen S. J., Bruyneel E. A., Bracke M. E., et al. Transition from the noninvasive to the invasive phenotype and loss of alpha-catenin in human colon cancer cells.// Cancer Res.-1995.-Vol. 55.-P.4722-4728.
21 Burns L. J., Pooley J. C., Walsh D. J., et al. Intercellular adhesion molecule-1 expression in endothelial cells is activated by cytomegalovirus immediate early proteins // Transplantation.-1999.-Vol. 67.- P. 137-144.
22 Cinatl J. Jr., Cinatl J., Vogel J. U., et al. Modulatory effects of human cytomegalovirus infection on malignant properties of cancer cells.// Intervirology.-1996.-Vol. 39.-P.259-269
23 US Patent application publication US2006/0063212 A1 Method of cancer screening; method of cancer treatment; and method of diabetes treatment.

1. A method of treatment of cancer patients that includes standard therapy, distinct in that an antiviral drug is used as an additional component of standard treatment before the beginning of and in parallel with standard treatment.

2. A method of treatment of cancer patients according to claim 1, distinct in that Valacyclovir is used as an antiviral drug.

3. A method of treatment of cancer patients according to claim 2, distinct in that the antiviral drug is administered in 3-7 courses of 7-20 days each, beginning treatment 1-5 days before the beginning of standard treatment and parallel with standard treatment in dosages of 1-2 g 3-4 times a day with intervals of 7-20 days each between courses.

4. A method of treatment of cancer patients according to claim 1, distinct in that the intravenously injectible form of Acyclovir is used as an antiviral drug.

5. A method of treatment of cancer patients according to claim 4, distinct in that Acyclovir is administered in 3-7 courses of 7-20 days each, beginning treatment 1-5 days before the beginning of standard treatment and parallel with standard treatment in dosages of 0.5-1 g 1-4 times a day with intervals of 7-20 days each between courses.

6. A method of treatment of cancer patients according to claim 1, distinct in that the injectible form of alpha interferon is used as an antiviral drug.

7. A method of treatment of cancer patients according to claim 1, distinct in that the injectible form of a specific antiviral immunoglobulin is used as an antiviral drug.

8. A method of treatment of cancer patients according to claim 1, distinct in that a combination of the drugs in claims 2-7 are used as antiviral drugs.

9. A method of treatment of cancer patients according to any one of claims 1, 3, and 5, distinct in that a method of surgical intervention, radiation therapy, chemotherapy, and their standardized combinations are used as standard therapy.
